(19) United States
(12) Patent Application Publication
(10) Pub. No.: US 2023/0130743 A1
(43) Pub. Date: Apr. 27, 2023

(54) Artificial Ankle Joint Bearing Element

(71) Applicant: Corentec Co., Ltd., Seoul (KR)

(72) Inventors: Keun-Bae Lee, Gwangju (KR); Jea-Won Kim, Seoul (KR); Sung-Wook Jung, Seoul (KR)

(21) Appl. No.: 17/915,384

(22) PCT Filed: Mar. 9, 2021
(86) PCT No.: PCT/KR2021/002930

§ 371 Date: Sep. 28, 2022

(30) Foreign Application Priority Data
Mar. 30, 2020 (KR) 10-2020-0038023

(51) Int. Cl. A61F 2/42

(52) U.S. Cl. CPC A61F 2/4202; A61F 2002/4205; A61F 2002/4207

(57) ABSTRACT

Proposed is an artificial ankle joint bearing element in which a contact area of the bearing element with a talus element is increased such that stress is evenly distributed during bearing movement of the bearing element on the talus element and wear of the bearing element is reduced under the same load; the bearing element has a front and a rear convexly formed to increase a contact area with a tibial element and distribute stress; and the front and rear of the bearing element are asymmetrically formed such that the rear thereof is formed to have a smaller height than the front thereof so as to facilitate the insertion of the bearing element into space between the talus element and the tibial element from an anterior side thereof during artificial ankle joint surgery.

### TECHNICAL FIELD

The present disclosure relates generally to an artificial ankle joint bearing element and, more specifically, to an artificial ankle joint bearing element in which a contact area of the bearing element with a talus element is increased such that stress is evenly distributed during bearing movement of the bearing element on the talus element and wear of the bearing element is reduced under the same load; the bearing element has a front and a rear convexly formed to increase a contact area with a tibial element and distribute stress; and the front and rear of the bearing element are asymmetrically formed such that the rear thereof is formed to have a smaller height than the front thereof so as to facilitate the insertion of the bearing element into space between the talus element and the tibial element from an anterior side thereof during artificial ankle joint surgery.

### BACKGROUND ART

Generally, when an ankle joint does not function properly due to various causes, such as degenerative arthritis and post-traumatic arthritis of an ankle, replacement using an artificial ankle joint is performed. Artificial ankle joint replacement, which was first started in the 1970s, shows lower-than-expected clinical results, including many side effects in the early stages, and the procedure of the replacement is highly complex, so artificial ankle joint replacement is a great burden to a practitioner and thus tends to be avoided, and thus is often replaced with ankle fixation. However, with the development of implants and the development of surgical methods, clinical results have gradually improved and patient satisfaction has increased, so artificial ankle joint replacement may be considered as joint replacement surgery which is commonly performed today.

There are several types of artificial ankle joints, and the artificial ankle joints are broadly divided into a tibial replacement coupled to a tibia, a talus replacement coupled to a talus, and a 3-component mobile bearing configured as a bearing element which functions as a bearing by connecting the two replacements to each other, and the mobile bearing is most widely used in Korea.

The prior art discloses an artificial ankle joint implant to replace an ankle joint, and particularly, provides a bearing between a tibial implant coupled to the distal end of a tibia and a talus implant coupled to the proximal end of a talus. First, the anatomical structure of an ankle in which the artificial ankle joint is implanted will be described with reference to FIG. 1. FIG. 1 is a side view illustrating a part of the ankle joint in which the distal end of a tibia 93 (a shinbone) is located (for convenience, a fibula is not shown). The tibia 93 is located on the upper side of a talus 91, and the talus 91 is located between the tibia 93, a scaphoid bone 95, and a calcaneus (a heel bone, 97). The tibia 93 performs dorsiflexion and plantar flexion while moving forward and rearward on a talar dome which is the proximal end of the talus 91. When performing the artificial ankle joint replacement, the dome of the talus 91 is cut to implant the talus implant therein, and a portion of the distal end of the tibia 93 is cut to implant the tibial implant therein, and the bearing element which functions as a bearing is inserted into space between the two implants 1 and 3, so the joint movement of an ankle is embodied. In the artificial ankle joint surgery performed in the prior art, as illustrated in FIG. 2, the size of the ankle is small compared to other joints, so the surgical portion of the ankle is very narrow. In addition, in the ankle joint replacement, a front approach method in which a front part of the ankle is mainly cut is used, and when approaching the ankle joint from an anterior side thereof, a cut range of the ankle is narrower. Accordingly, it is difficult to check a cut portion during surgery, and after implanting the tibial implant and the talus implant from the anterior side of the ankle, the bearing element is inserted into space between the tibial implant and the talus implant from the anterior side.

### Patent Document

European Patent Application publication No. EP 1731115 A1 “Cement-free tibial component for an ankle replacement prosthesis and an ankle prosthesis comprising such a component”.

As illustrated in FIG. 3, the cement-free tibial component illustrated in the patent document includes a tibial element 71 suitable for being attached to a tibia, a double bearing element 73, and a talus element 75 or plate suitable for being attached to a talus of the foot so as to embody the natural movement of an ankle joint. An artificial ankle joint bearing element which is also called the double bearing element can embody the dorsiflexion and plantar flexion of the ankle joint by providing a bearing between the tibial element 71 and the talus element 75.

FIG. 4 is a bottom view of the bearing element of the prior art seen from a lower side thereof. Referring to FIG. 4, the bearing element of the prior art includes a bearing surface 731 provided to be in contact with the talus element while the bearing element slides on the talus element 75, the bearing surface protruding downward gradually toward the center of the bearing element from inner and outer sides thereof, a front connection surface 733 provided in front of the bearing surface 731, and a rear connection surface 735 provided behind the bearing surface 731. In the bearing element 73 of the prior art, the front connection surface 733 and the rear connection surface 735 protrude convexly downward, but the bearing element 73 is designed to be flat as a whole, so the bearing element 73 cannot make extensive contact with the talus element attached to a talus. When contact between the bearing element and the talus element is made in a narrow range, stress is concentrated, so the bearing element is broken or the amount of wear of the bearing element is increased. In addition, since the front connection surface 733 and the rear connection surface 735 are designed to be flat, a load transmitted from the tibial element 71 cannot be sufficiently distributed.

FIG. 5 is a side view of the bearing element of the prior art. The bearing element of the prior art has front and rear portions designed symmetrically to each other. In actual surgery, the bearing element 73 is inserted from the anterior side as illustrated in FIG. 6, and a vertical height H2 between a surface on which the rear portion of the bearing element 73 is in contact with the tibial element 71 and the lowermost end of the bearing surface 731 are equal to a vertical height H1 between a surface on which the front portion of the bearing element 73 is in contact with the tibial element 71 and the lowermost end of the bearing surface 731, so since the bearing element is interfered during the insertion of the bearing element, the stable insertion and fixing of the bearing element are difficult, and when the bearing element is forcibly inserted, the bearing element may scratch or damage the articular surface of the talus element 75 or the tibial element 71.

Accordingly, required is an artificial ankle joint bearing element in which during the movement of an ankle joint such as buckling or dorsiflexion of an artificial ankle joint, a contact area of the bearing element with the talus element is increased to evenly distribute stress and reduce wear of the bearing element under the same load, and when the artificial ankle joint replacement is performed, the insertion of the bearing element into an ankle is easy.

### DISCLOSURE

### Technical Problem

The present disclosure has been made to solve the above problems, and
the present disclosure is intended to provide an implant, which is a bearing element, which embodies the movement of an ankle between a talus element and a tibial element, the bearing element including: a bearing surface which enables the movement of the bearing element on the articular surface of the talus element while the bearing element is in contact with the talus element; a peripheral surface formed to surround the lateral surface of the bearing element; and a connection surface which connects the peripheral surface with the bearing surface, wherein the connection surface includes a front connection surface formed on the front of the bearing element and a rear connection surface formed on the rear thereof, and each of the front connection surface and the rear connection surface is formed by having a predetermined angle from the peripheral surface, and the front connection surface is formed to have curvature and includes a front inner connection surface bordered by an inner bearing surface and a front outer connection surface bordered by an outer bearing surface, wherein the front inner connection surface and the front outer connection surface extend respectively from the inner and outer sides of the front connection surface such that the center of the front inner connection surface and the front outer connection surface protrudes forward, and thus the contact area of the bearing surface with the talus element is increased, so the bearing element evenly distributes stress and has excellent wear resistance.

In addition, the present disclosure is intended to provide an implant in which the front inner connection surface and the front outer connection surface are formed by having curvatures and extend respectively from the inner and outer sides of the front connection surface such that the center of the front inner connection surface and the front outer connection surface protrudes by having curvature, so that the front inner connection surface and the front outer connection surface have a shape corresponding to the shape of the upper surface of the talus element, whereby the bearing element can stably slide on the talus element.

Furthermore, the present disclosure is intended to provide an implant in which the rear connection surface is formed to have curvature and includes a rear inner connection surface bordered by the inner bearing surface and a rear outer connection surface bordered by the outer bearing surface, wherein the rear inner connection surface and the rear outer connection surface extend respectively from the inner and outer sides of the rear connection surface such that the center of the inner bearing surface and the outer bearing surface protrudes rearward, and thus the contact area of the bearing surface with the talus element is increased, so the implant evenly distributes stress and has excellent wear resistance.

Additionally, the present disclosure is intended to provide an implant in which a tibial element contact surface extends convexly forward and rearward such that a load transmitted from the tibial element can be effectively distributed.

In addition, the present disclosure is intended to provide an implant in which the front and rear of the implant are formed asymmetrically so as to facilitate the insertion of the implant during surgery.

Furthermore, the present disclosure is intended to provide an implant in which height between the tibial element contact surface and the lowermost end of the rear connection surface is smaller than height between the tibial element contact surface and the lowermost end of the front connection surface, so that the insertion of the implant from an anterior side is facilitated and the convenience of surgery is increased.

### Technical Solution

In order to accomplish the above objectives, the present disclosure is embodied by embodiments of an artificial ankle joint bearing element having the following components.

According to an embodiment of the present disclosure, the artificial ankle joint bearing element of the present disclosure allows the movement of an ankle to be embodied between a talus element and a tibial element and includes: a bearing surface which enables the movement of the bearing element on an articular surface of the talus element while the bearing element is in contact with the talus element; a peripheral surface formed to surround a lateral surface of the bearing element; and a connection surface which connects the peripheral surface with the bearing surface.

According to another embodiment of the present disclosure, in the artificial ankle joint bearing element of the present disclosure, the connection surface may include a front connection surface formed on the front of the bearing element and a rear connection surface formed on the rear of the bearing element, wherein each of the front connection surface and the rear connection surface may be formed by having a predetermined angle from the peripheral surface.

According to still another embodiment of the present disclosure, in the artificial ankle joint bearing element of the present disclosure, the front connection surface may include: a front inner connection surface bordered by an inner bearing surface and a front outer connection surface bordered by an outer bearing surface, wherein the front inner connection surface and the front outer connection surface may extend respectively from inner and outer sides of the front connection surface such that a center of the front inner connection surface and the front outer connection surface protrudes forward, so that a contact area of the bearing surface with the talus element may be increased.

According to still another embodiment of the present disclosure, in the artificial ankle joint bearing element of the present disclosure, the front inner connection surface and the front outer connection surface may be famed by having curvatures and extend respectively from the inner and outer sides of the front connection surface such that the center of the front inner connection surface and the front outer connection surface protrudes by having a curved surface.

According to still another embodiment of the present disclosure, in the artificial ankle joint bearing element of the present disclosure, the rear connection surface may include a rear inner connection surface bordered by the inner bearing surface and a rear outer connection surface bordered by the outer bearing surface, wherein the rear inner connection surface and the rear outer connection surface may extend respectively from inner and outer sides of the rear connection surface such that a center of the rear inner connection surface and the rear outer connection surface protrudes rearward, so that the contact area of the bearing surface with the talus element may be increased.

According to still another embodiment of the present disclosure, in the artificial ankle joint bearing element of the present disclosure, the rear inner connection surface and the rear outer connection surface may be famed by having curvatures and extend respectively from the inner and outer sides of the rear connection surface such that the center of the rear inner connection surface and the rear outer connection surface protrudes by having a curved surface.

According to still another embodiment of the present disclosure, the artificial ankle joint bearing element of the present disclosure may further include a tibial element contact surface formed to be in contact with the tibial element, and the peripheral surface may include a front surface formed on a front of the bearing element and a rear surface formed on a rear of the bearing element.

According to still another embodiment of the present disclosure, in the artificial ankle joint bearing element of the present disclosure, a front boundary which foams a boundary between the front surface and the tibial element contact surface may be formed in a form of an arc by being convex forward, and the tibial element contact surface may be formed by extending forward up to the front boundary.

According to still another embodiment of the present disclosure, in the artificial ankle joint bearing element of the present disclosure, a rear boundary which forms a boundary between the rear surface and the tibial element contact surface may be formed in a form of an arc by being convex rearward, and the tibial element contact surface may be formed by extending rearward up to the rear boundary.

According to still another embodiment of the present disclosure, the artificial ankle joint bearing element of the present disclosure may include: the bearing surface which enables the movement of the bearing element on the articular surface of the talus element while the bearing element is in contact with the talus element; the peripheral surface formed to surround the lateral surface of the bearing element; the connection surface which connects the peripheral surface with the bearing surface; and the tibial element contact surface formed to be in contact with the tibial element, wherein the connection surface may include the front connection surface formed on the front of the bearing element and the rear connection surface formed on the rear of the bearing element, and each of the front connection surface and the rear connection surface may be formed by having a predetermined angle from the peripheral surface, and the front and rear of the bearing element may be formed asymmetrically.

According to still another embodiment of the present disclosure, in the artificial ankle joint bearing element of the present disclosure, height between the tibial element contact surface and the lowermost end of the front connection surface may be different from height between the tibial element contact surface and the lowermost end of the rear connection surface.

According to still another embodiment of the present disclosure, in the artificial ankle joint bearing element of the present disclosure, height between the tibial element contact surface and the lowermost end of the rear connection surface may be smaller than height between the tibial element contact surface and the lowermost end of the front connection surface.

According to still another embodiment of the present disclosure, the artificial ankle joint bearing element of the present disclosure may further include: a front bearing boundary which is formed on a lower end of the front connection surface and forms a boundary between the bearing surface and the front connection surface; and a rear bearing boundary which is formed on a lower end of the rear connection surface and forms a boundary between the bearing surface and the rear connection surface.

According to still another embodiment of the present disclosure, in the artificial ankle joint bearing element of the present disclosure, the front bearing boundary may include: a front inner bearing boundary which forms a boundary between the front connection surface and an inner bearing surface; and a front outer bearing boundary which forms a boundary between the front connection surface and an outer bearing surface, wherein the front inner bearing boundary and the front outer bearing boundary may extend respectively from inner and outer sides of the front bearing boundary such that a center of the front inner bearing boundary and the front outer bearing boundary protrudes downward to form the lowermost end of the front connection surface, and the rear bearing boundary may include: a rear inner bearing boundary which forms a boundary between the rear connection surface and the inner bearing surface; and a rear outer bearing boundary which forms a boundary between the rear connection surface and the outer bearing surface, wherein the rear inner bearing boundary and the rear outer bearing boundary may extend respectively from inner and outer sides of the rear bearing boundary such that a center of the rear inner bearing boundary and the rear outer bearing boundary protrudes downward to form the lowermost end of the rear connection surface.

According to still another embodiment of the present disclosure, in the artificial ankle joint bearing element of the present disclosure, the peripheral surface may include: the front surface formed on the front of the bearing element; and the rear surface formed on the rear of the bearing element,
wherein a height of the front surface extending vertically from the tibial element contact surface may be the same as a height of the rear surface extending vertically from the tibial element contact surface, and height between a boundary between the rear surface and the rear connection surface and the lowermost end of the rear connection surface may be smaller than height between a boundary between the front surface and the front connection surface and the lowermost end of the front connection surface.

According to still another embodiment of the present disclosure, in the artificial ankle joint bearing element of the present disclosure, the front connection surface may include: the front inner connection surface bordered by the inner bearing surface; and the front outer connection surface bordered by the outer bearing surface, wherein the front inner connection surface and the front outer connection surface may extend respectively from the inner and outer sides of the front connection surface such that the center of the front inner connection surface and the front outer connection surface protrudes forward, and the rear connection surface may include: the rear inner connection surface bordered by the inner bearing surface, and the rear outer connection surface bordered by the outer bearing surface, wherein the rear inner connection surface and the rear outer connection surface extend respectively from the inner and outer sides of the rear connection surface such that the center of the rear inner connection surface and the rear outer connection surface protrudes rearward, so that the contact area of the bearing surface with the talus element may be increased.

### Advantageous Effects

The present disclosure can obtain the following effects by the above described embodiments, and components of the bearing element, combination thereof, and use relationship thereof which will be described below.

According to the present disclosure, the bearing element enables the movement of an ankle between the talus element and the tibial element, and includes the bearing surface which enables the movement of the bearing element on the articular surface of the talus element while the bearing element is in contact with the talus element, the peripheral surface formed to surround the lateral surface of the bearing element, and the connection surface which connects the peripheral surface with the bearing surface, wherein the connection surface includes the front connection surface formed on the front of the bearing element and the rear connection surface formed on the rear of the bearing element, and each of the front connection surface and the rear connection surface is formed by having a predetermined angle from the peripheral surface, and the front connection surface is formed to have curvature and includes the front inner connection surface bordered by the inner bearing surface and the front outer connection surface bordered by the outer bearing surface, wherein the front inner connection surface and the front outer connection surface extend respectively from the inner and outer sides of the front connection surface such that the center of the front inner connection surface and the front outer connection surface protrudes forward, and thus the contact area of the bearing surface with the talus element is increased, thereby evenly distributing stress and having excellent wear resistance.

In addition, according to the present disclosure, the front inner connection surface and the front outer connection surface are formed by having curvatures and extend respectively from the inner and outer sides of the front connection surface such that the center of the front inner connection surface and the front outer connection surface protrudes by having a curved surface, so that the front inner connection surface and the front outer connection surface have a shape corresponding to the shape of the upper surface of the talus element, thereby enabling the bearing element to stably slide on the talus element.

In addition, according to the present disclosure, the rear connection surface is formed to have curvature and includes the rear inner connection surface bordered by the inner bearing surface and the rear outer connection surface bordered by the outer bearing surface, wherein the rear inner connection surface and the rear outer connection surface extend respectively from the inner and outer sides of the rear connection surface such that the center of the rear inner connection surface and the rear outer connection surface protrudes rearward, and thus the contact area of the bearing surface with the talus element is increased, thereby allowing the bearing element to evenly distribute stress and to have excellent wear resistance.

In addition, according to the present disclosure, the tibial element contact surface extends convexly forward and rearward, thereby effectively distributing a load transmitted from the tibial element.

In addition, according to the present disclosure, the front and rear of the bearing element are formed asymmetrically, thereby facilitating the insertion of the bearing element during surgery.

In addition, according to the present disclosure, height between the tibial element contact surface and the lowermost end of the rear connection surface is smaller than height between the tibial element contact surface and the lowermost end of the front connection surface, thereby facilitating the insertion of an implant from the anterior side and increasing convenience of surgery.

### DESCRIPTION OF DRAWINGS

FIG. 1 is a side view illustrating portions of an ankle joint in which the proximal end of a talus 91 and a distal end of a tibia 93 (shinbone) are located.

FIG. 2 illustrates a scene in which artificial ankle joint replacement is performed in an operating room.

FIG. 3 is a perspective view of a 3-component artificial ankle joint of a prior art.

FIG. 4 is a bottom view of a bearing element of the prior art seen from a lower side thereof.

FIG. 5 is a side view of the bearing element of the prior art.

FIG. 6 is a view illustrating the insertion of the bearing element, which is a tibial insert of the artificial ankle joint, from an anterior side.

FIG. 7 is a bottom perspective view of an artificial ankle joint bearing element 5 according to an exemplary embodiment of the present disclosure.

FIG. 8 is a bottom view of the artificial ankle joint bearing element 5 according to the exemplary embodiment of the present disclosure.

FIG. 9 is an upper perspective view of the artificial ankle joint bearing element 5 according to the embodiment of the present disclosure.

FIG. 10 is a top view of the artificial ankle joint bearing element 5 according to the exemplary embodiment of the present disclosure.

FIG. 11 is a side view of the artificial ankle joint bearing element 5 according to the exemplary embodiment of the present disclosure.

FIG. 12 is a view illustrating heights of the front and rear of the bearing element and an angle between a peripheral surface 55 and each of a front connection surface 571 and a rear connection surface 573 in the side view of the artificial ankle joint bearing element 5 according to the exemplary embodiment of the present disclosure.

FIG. 13 illustrates the movement of each of the bearing element of the prior art and the bearing element 5 according to the exemplary embodiment of the present disclosure on the upper surface of the talus element 1 while performing bearing action.

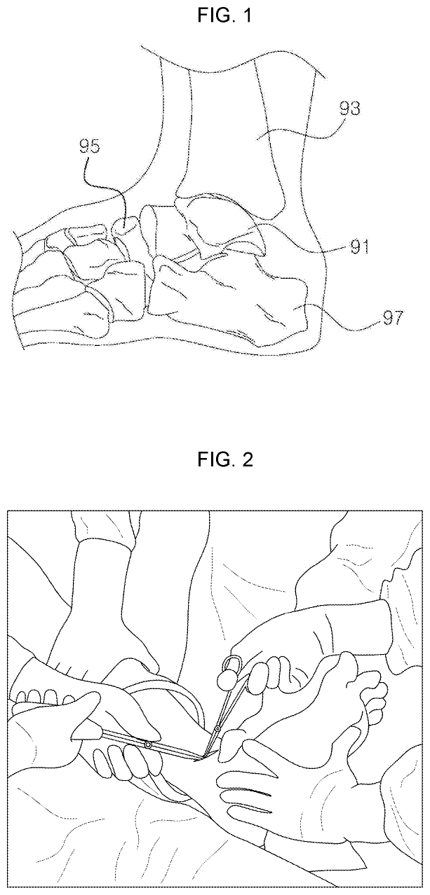

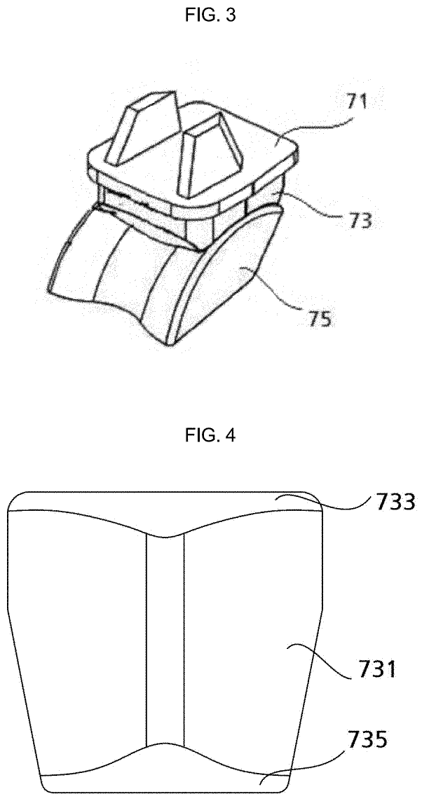

### BEST MODE

Hereinafter, an artificial ankle joint bearing element of the present disclosure will be described in detail with reference to the accompanying drawings. It should be noted that the same components in the drawings are denoted by the same reference numerals wherever possible. In addition, the detailed description of known function and configuration that may unnecessarily obscure the gist of the present disclosure will be omitted. Unless otherwise defined, all terms in this specification have the same meaning as the general meaning of terms understood by those skilled in the art to which the present disclosure belongs, and when the terms conflict with the meaning of the terms used in this specification, the terms used in this specification has higher priority.

Now, the artificial ankle joint the talus element of the present disclosure will be described in detail with reference to the accompanying drawings.

FIG. 6 is a view illustrating the insertion of the bearing element which is a tibial insert of the artificial ankle joint from an anterior side. An artificial ankle joint system, in which the talus element 1, a tibial element 3, and the bearing element 5 are coupled to each other to replace the movement of an ankle joint, and a principle thereof will be briefly described with reference to FIG. 6.

The bearing element 5 made of plastic is located on the talus element 1 and performs the function of a bearing, and the talus element 1 reproduces a joint movement corresponding to dorsiflexion and plantar flexion while sliding forward and rearward along the curvature of the lower surface of the bearing element 5 due to the movement of an ankle. The tibial element 3 is located on the bearing element 5 and is coupled to the distal end of a tibia 93 and receives a load from the tibia 93. The tibial element 3 may be a fixed element which is completely coupled to the bearing element 5, may be a semi-fixed element such that the tibial element 3 and the bearing element 5 partially restrain each other to perform limited relative movements, or may be a free element whose free movement is possible. In place of an ankle, these three elements are coupled to each other to perform a joint movement.

First, referring to FIG. 6, the talus element 1 of the artificial ankle joint will be roughly described. The talus element 1 reproduces a joint movement corresponding to dorsiflexion and plantar flexion while sliding forward and rearward along the curvature of the lower surface of the bearing element 5 due to the movement of the ankle. The upper surface of the talus element 1, that is, a part thereof which is in contact with the bearing element 5 to embody a joint movement is formed to be curved by having curvature in a front-to-rear direction to guide the joint movement of the bearing element 5, and due to the configuration of the curvature, the dorsiflexion of raising an ankle up and the plantar flexion of bending the ankle downward are facilitated after the artificial ankle joint surgery. For more natural movement of the ankle, the curvature is preferably preset to be similar to the curvature of an actual talar dome. In addition, in the part which embodies the joint movement by being in contact with the bearing element 5, a connection surface between the inner and outer surfaces of the talus element is formed by being depressed, and due to such a shape, when the bearing element 5 performs a joint movement forward and rearward along the talus element 1, the bearing element 5 can stably move without being removed to inner and outer sides.

The artificial ankle joint bearing element 5 according to the exemplary embodiment of the present disclosure increases a contact area with the talus element 1 to evenly distribute stress during the bearing movement of the bearing element 5 on the talus element 1 and to reduce wear of the bearing element under the same load. The bearing element 5 is convexly formed in front and rear thereof and increases a contact area with the tibial element 3 to distribute stress, and the front and rear are formed asymmetrically to each other such that the height of the rear is smaller than the height of the front, so during artificial ankle joint surgery, the bearing element is easily inserted into space between the talus element 1 and the tibial element 3 from the anterior side thereof. The bearing element 5 includes the bearing surface 51, a tibial element contact surface 53, a peripheral surface 55, and the connection surface 57, and a front bearing boundary 58 is formed between the bearing surface 51 and a front connection surface 571 to be described later, and a rear bearing boundary 59 is formed between the bearing surface 51 and a rear connection surface 573 to be described later.

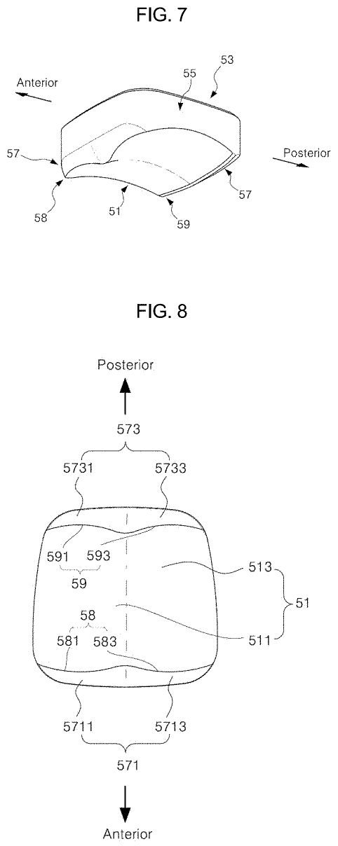

FIG. 7 is a bottom perspective view of the artificial ankle joint bearing element 5 according to the exemplary embodiment of the present disclosure, and FIG. 8 is a bottom view of the artificial ankle joint bearing element 5 according to the exemplary embodiment of the present disclosure. Referring to FIGS. 7 and 8, the bearing surface 51 enables the movement of the bearing element on the articular surface of the talus element while the bearing element is in contact with the talus element. The bearing element 5 slides on the upper surface of the talus element 1 described above while the bearing surface 51 is in contact with the upper surface of the talus element 1 so as to embody the movement of an ankle. Accordingly, the bearing surface 51 may be formed to have a shape corresponding to the upper surface of the talus element 1. The bearing surface 51 may be formed in an angular shape, but is preferably formed in a gently curved shape. As in the prior art, when each of the inner and outer boundaries of the bearing element 5 or the talus element 1 is formed as high as a predetermined length, the effect of preventing the removal of the bearing element 5 is excellent, but when the bearing element 5 is removed, it is impossible to restore the bearing element 5, so the restoration of the bearing element 5 is inevitably performed by a surgical method. However, when the boundaries of the bearing element 5 have curved shapes, the bearing element 5 can be naturally restored to an initial position thereof even if the removal of the bearing element 5 occurs. The bearing surface 51 includes an inner bearing surface 511 and an outer bearing surface 513.

The inner bearing surface 511 is defined as a bearing surface formed on a portion close to the center of a human body relative to an AP line connected from the anterior side to the posterior side, which crosses the center of the bearing element 5, and the outer bearing surface 513 is defined as a bearing surface formed on a portion close to the outer side of a human body relative to the AP line. The center portion of the inner bearing surface 511 and the outer bearing surface 513 may extend to protrude relatively downward. The inner bearing surface 511 and the outer bearing surface 513 are provided to be connected to each other in the center portion.

In the exemplary embodiment of the present disclosure, as illustrated in FIGS. 7 and 8, when the inner bearing surface 511 extends to the center portion of the bearing surface from the inner side thereof, the inner bearing surface 511 extends downward by having a curved surface, and when the outer bearing surface 513 extends to the center portion of the bearing surface from the outer side thereof, the outer bearing surface 513 extends downward by having a curved surface, so the center portion of the bearing surface 51 may be formed to protrude downward by having a gently curved surface. Accordingly, the bearing surface 51 has a shape approximately corresponding to the talus element 1 described above, and thus the bearing element 5 can reproduce the movement of an ankle joint by sliding on the talus element 1. In another embodiment of the present disclosure, when the inner bearing surface 511 and the outer bearing surface 513 extend to the center portion of the bearing surface from the inner and outer sides thereof, respectively, the inner bearing surface 511 and the outer bearing surface 513 may be connected to each other in such a manner that the center portion of the bearing surface is not formed as a curved surface but has a predetermined angle.

Referring to FIG. 8, the bearing surface 51 may have a shape widening gradually toward the anterior side. A talus 91 has a shape wider in a front thereof than in a rear thereof, and when the talus element 1 has a complementary shape to the cut surface of the talus 91, the talus element 1 may have a truncated cone-shaped structure wider in a front thereof than in a rear thereof as a whole. The bearing element 5 also has a shape wider in the front thereof than in the rear thereof to correspond to the shape of this talus element 1 so as to maximize the movable range of the bearing element 5 and to evenly distribute stress transmitted to the talus element 1, thereby improving the life of the artificial ankle joint. Further, the bearing element 5 may be designed closer to the anatomical shape of the ankle joint to be a more physiological joint implant, thereby providing a comfortable joint after surgery.

The bearing surface 51 may be formed to protrude downward gradually toward the anterior and posterior sides of the bearing surface from a center thereof by having a upward concave shape. Accordingly, the bearing element 5 may slide on the upper surface of the talus element 1, which is relatively convexly formed in a center portion thereof, so as to reproduce the movement of an ankle joint. In this case, as described later, the anterior and posterior sides of the bearing surface 51 are asymmetrically formed such that the insertion of the bearing surface 51 is facilitated during surgery.

In addition, the bearing surface 51 is bordered by the peripheral surface 55 and the connection surface 57 which will be described later, and preferably extends forward, rearward, inward, and outward by having a curved shape with curvature up to an associated boundary. The bearing surface 51 extends by having a curved shape, and thus a boundary between the bearing surface 51 and another surface may also be formed in the form of a curved line. Particularly, hereinafter, a boundary between the bearing surface 51 and the front connection surface 571 to be described later is defined as the front bearing boundary 58, and a boundary between the bearing surface 51 and the rear connection surface 573 is defined as the rear bearing boundary 59.

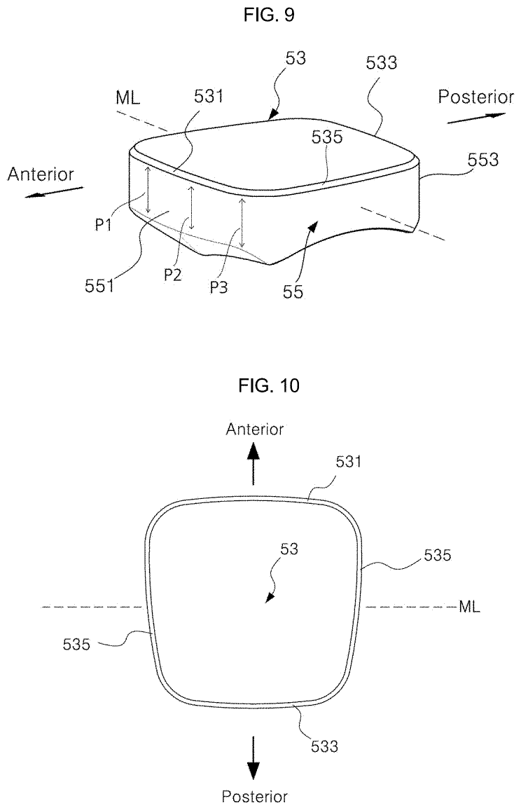

FIG. 9 is the upper perspective view of the artificial ankle joint bearing element 5 according to the embodiment of the present disclosure, and FIG. 10 is a top view of the artificial ankle joint bearing element 5 according to the exemplary embodiment of the present disclosure. Referring to FIGS. 9 and 10, the tibial element contact surface 53 is in contact with the tibial element 3 and is formed to support a load transmitted from the tibial element 3 and distribute stress. In the case of the restraining artificial ankle joint, the tibial element 3 and the bearing element 5 are integrated with each other and the tibial element contact surface 53 and the lower surface of the tibial element 3 are completely coupled to each other. The tibial element contact surface 53 is formed to be approximately flat and is bordered by the peripheral surface 55 to be described later, and includes a front boundary 531, a rear boundary 533, a lateral boundary 535.

The front boundary 531 is configured to form a border between a front surface 551 formed on the front of the peripheral surface 55 to be described later and the tibial element contact surface 53. The front boundary 531 may be formed in the form of an arc by being convex forward. Since the front boundary 531 is formed in the form of an arc by being convex forward, the flat surface of the tibial element contact surface 53 may have a shape protruding convexly forward. As illustrated in FIG. 4, the bearing element of the prior art is formed to have front and rear which are flat or straight, and thus does not effectively distribute stress transmitted from the tibial element 3, so the bearing element of the prior art is worn and broken. In contrast, the bearing element 5 according to the present disclosure has the tibial element contact surface 53 protruding forward, and thus can evenly distribute stress and can have better wear resistance under the same load.

The rear boundary 533 is configured to form a boundary between a rear surface 553 formed on the rear of the peripheral surface 55 to be described later and the tibial element contact surface 53. The rear boundary 533 may be formed in the form of an arc by being convex rearward. Since the rear boundary 533 is formed in the form of an arc by being convex rearward, the flat surface of the tibial element contact surface 53 may have a shape protruding convexly rearward. Similarly to the case of the front boundary 531 described above, the bearing element 5 according to the present disclosure has the tibial element contact surface 53 protruding rearward, and thus can evenly distribute stress and can have better wear resistance under the same load.

The lateral boundary 535 is configured to form a boundary between each of the inner and outer sides of the peripheral surface 55 to be described later and the tibial element contact surface 53. The lateral boundary 535 may be formed to have the shape of a gently curved line, and is preferably formed to correspond to the lower side surface of the tibial element 3 to effectively distribute stress transmitted from the tibial element 3 and is preferably formed for the bearing element 5 to efficiently slide on the talus element 1.

Referring to FIG. 10, the lateral boundary 535 may be formed on each of the inner and outer sides of the tibial element contact surface 53 such that a distance between the lateral boundaries 535 is increased gradually toward the anterior side. Accordingly, the tibial element contact surface 53 may have a shape widening gradually toward the anterior side. The tibia 93 has a structure in which a front of the tibia 93 is wider than a rear thereof, and when the tibial element 3 has a shape complementary to the cut surface of the tibia 93, the tibial element 3 may have a structure in which the front of the tibial element 3 is wider than the rear thereof. The bearing element 5 has a shape corresponding to the shape of the tibial element 3 so as to evenly distribute stress transmitted to the bearing element 5, thereby increasing the life of an artificial ankle joint, and is designed closer to the anatomical shape of the ankle joint to be a more physiological joint implant, thereby providing a comfortable joint after surgery.

Referring back to FIG. 9, the peripheral surface 55 is formed to surround the side surface of the bearing element 5 according to the present disclosure. The peripheral surface 55 can constitute the four side surfaces of the front, rear, inner side, and outer side of the bearing element 5 by extending approximately vertically downward from the front boundary 531, the rear boundary 533, and the lateral boundary 535 bordered by the tibial element contact surface 53. The heights of points of the peripheral surface 55 extending from the tibial element contact surface 53 may be different from each other. The peripheral surface 55 includes the front surface 551 and the rear surface 553.

The front surface 551 is formed on the front of the bearing element 5 by extending vertically downward from the front boundary 531. Since the front boundary 531 is formed in the form of an arc by being convex forward, the front surface 551 may also have the shape of a curved surface convex forward.

The rear surface 553 is formed on the rear of the bearing element 5 and extends approximately vertically downward from the rear boundary 533. Since the rear boundary 533 is formed in the form of an arc by being convex rearward, the rear surface 553 may have the shape of a curved surface which is convex rearward.

As described above, heights of the peripheral surface 55 extending downward from points of the boundaries may be different from each other, and particularly, the heights of the front surface 551 and the rear surface 553 may be important. In the exemplary embodiment of the present disclosure, the front surface 551 may extend vertically downward from points of the front boundary 531 by having approximately same heights, and the rear surface 553 may extend vertically downward from the rear boundary 533 by having approximately same heights. The downward vertical extension of the front surface 551 from the points of the front boundary by having the same heights means that heights between the points of the front boundary 531 and the upper end of the front connection surface 571 to be described later are the same (P1=P2). However, in another embodiment of the present disclosure, the front surface 551 and the rear surface 553 may extend respectively from the front boundary 531 and the rear boundary 533 by having different heights (P1≠P3). In this case, each of the front surface and the rear surface may extend symmetrically in an inside-to-outside direction.

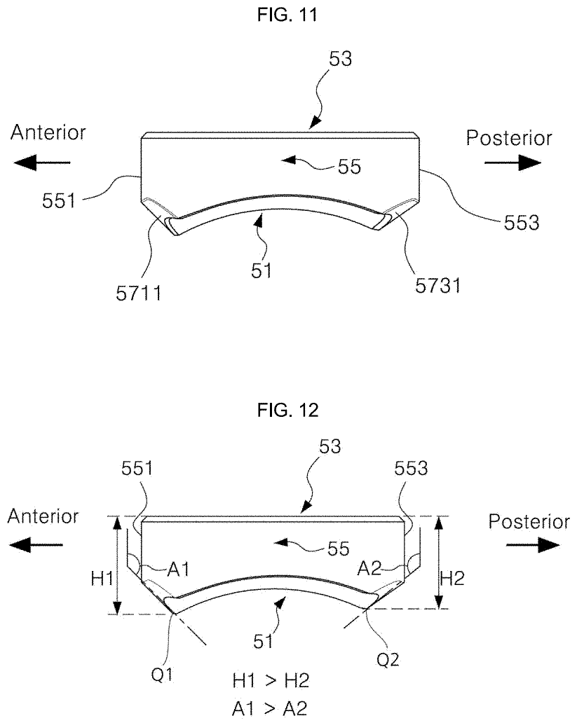

FIG. 11 is a side view of the artificial ankle joint bearing element 5 according to the exemplary embodiment of the present disclosure, and FIG. 12 is a view illustrating heights of the front and rear of the bearing element and an angle between the peripheral surface 55 and each of the front connection surface 571 and the rear connection surface 573 in the side view of the artificial ankle joint bearing element 5 according to the exemplary embodiment of the present disclosure. Referring to FIGS. 11 and 12, the front and rear of the bearing element 5 may be formed asymmetrical to each other. Specifically, the height H2 of the rear may be lower than the height H1 of the front (H1>H2). In this case, the rear surface 553 may be formed to have an extending height lower than the extending height of the front surface 551, but the extending height of the rear surface 553 and the extending height of the front surface 551 is preferably the same, and the front connection surface 571 and the rear connection surface 573 which will be described later may be formed to have different extending heights.

In this case, each of the heights H1 and H2 of the front and the rear indicates height from the tibial element contact surface 53, and is defined as a vertical distance from the tibial element contact surface 53 which is approximately flat. Accordingly, as illustrated in FIG. 12, height between the lowermost end of the front connection surface 571 and the tibial element contact surface 53 may be defined as H1 in a vertical direction from the tibial element contact surface 53, and height between the lowermost end of the rear connection surface 573 and the tibial element contact surface 53 may be defined as H2.

Referring back to FIGS. 7 and 8, the connection surface 57 is formed between the bearing surface 51 and the peripheral surface 55 described above and is formed to connect the bearing surface 51 with the peripheral surface 55 in the lower front and rear of the bearing element 5. The bearing surface 51 is formed in a shape approximately corresponding to the upper surface of the talus element 1, and the peripheral surface 55 extends approximately vertically from the boundary of the tibial element contact surface 53. For the collision prevention and efficient movement of the bearing element 5, the connection surface 57 is formed between the bearing surface 51 and each of the front surface 551 and the rear surface 553. The peripheral surface 55 extends approximately vertically downward from the front boundary 531, the rear boundary 533, and the lateral boundary 535 of the tibial element contact surface 53, but the connection surface 57 may be formed as a curved surface with a predetermined curvature by having a predetermined angle from the peripheral surface 55. Specifically, the connection surface 57 is formed by having a predetermined angle from the front surface 551 or the rear surface 553. The connection surface 57 includes the front connection surface 571 and the rear connection surface 573.

Referring to FIGS. 7 and 8, the front connection surface 571 is formed on the front of the bearing element 5 so as to connect the front surface 551 with the bearing surface 51, and is formed to have a predetermined angle A1 from the front surface 551 of the peripheral surface 55 by having a predetermined curvature. The front connection surface 571 includes a front inner connection surface 5711 and a front outer connection surface 5713.

The front inner connection surface 5711 is bordered by the inner bearing surface 511 through the front bearing boundary 58 to be described later. The inner bearing surface 511 indicates a bearing surface formed on a portion close to the center of a human body relative to the AP line crossing the center of the bearing element 5, so the front inner connection surface 5711 is defined as a front connection surface formed on a portion close to the center of a human body.

The front outer connection surface 5713 is bordered by the outer bearing surface 513 through the front bearing boundary 58. Accordingly, the front outer connection surface 5713 indicates a front connection surface formed on a portion close to the outer side of a human body relative to the AP line.

The front inner connection surface 5711 and the front outer connection surface 5713 extend respectively from the inner and outer sides of the front connection surface to the center portion of the bearing element 5 on which the AP line thereof is formed so as to be connected with each other in the center portion. In this case, the center portion on which the front inner connection surface 5711 and the front outer connection surface 5713 are connected with each other may be formed to protrude forward. As illustrated in FIGS. 11 and 12, it can be seen that when viewing the bearing element 5 from a side, the center portion protrudes forward more than the inner side of the front inner connection surface 5711. In the exemplary embodiment of the present disclosure, the bearing element 5 may be formed symmetrically in a left-to-right direction, that is, in the inside-to-outside direction, so the center portion is also formed to protrude forward more than the outer side of the front outer connection surface 5713. The center portion of the front connection surface 571 is formed to protrude forward, and thus the contact area of the bearing element 5 with the talus element 1 is increased such that the bearing element distributes stress, so the bearing element 5 is prevented from being easily broken and has improved wear resistance.

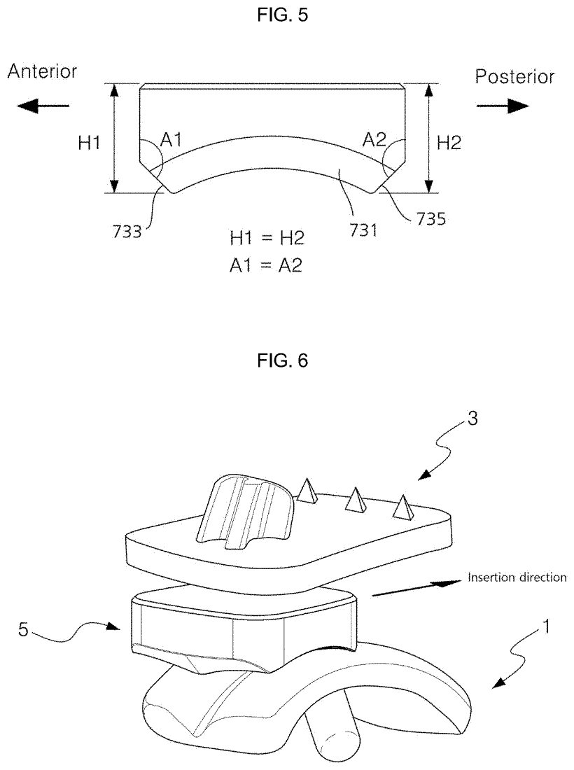

Referring to the side view of the prior art of FIG. 5, in the case of the prior art, it can be seen that the center portion of the connection surface of the front or rear side is formed to be flat without protruding. In this case, during the movement of the artificial ankle joint, the contact area of the bearing element with the talus element is not sufficiently wide, and it is difficult to effectively distribute stress, and accordingly, each element constituting the artificial ankle joint is broken or the amount of wear thereof is increased. In contrast, the center portion of each of the front connection surface 571 and the rear connection surface 573 to be described later according to the present disclosure protrudes forward and thus the contact area of the bearing element with the talus element 1 is maximized such that stress can be effectively distributed and wear of the bearing element can be prevented.

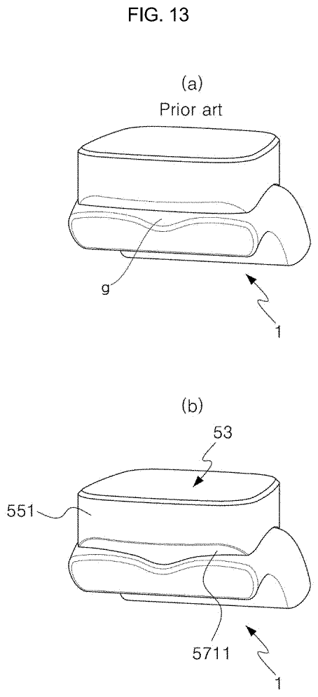

In order to explain this in more detail, referring to FIG. 13(b), it can be seen that when the center portion of the front connection surface 571 protrudes forward, the contact area of the bearing element with the talus element 1 is increased. When considering the shape of the upper surface of the talus element 1, the upper surface of the talus element is depressed in a center portion thereof, and in the case of the prior art, the front connection surface is provided to be flat, and thus even if the bearing element moves to the front end of the talus element on the talus element, a gap g occurs between the depressed center portion and the bearing element. In contrast, in the case of FIG. 13(b), the center portion of the front connection surface 571 protrudes forward, and thus can be in contact with the upper surface of the talus element 1 without a gap therebetween, and accordingly, the contact area of the bearing element with the talus element 1 is increased. Particularly, when the shape of the curved surface of the front connection surface 571 is formed to correspond to the front boundary of the upper surface of the talus element 1, the contact of the bearing surface 51 with the talus element 1 can be further increased.

In the exemplary embodiment of the present disclosure, when the front inner connection surface 5711 extends from the inner side of the front connection surface 571 to the center portion thereof, the front inner connection surface 5711 extends forward while forming a curved surface, and when the front outer connection surface 5713 extends from the outer side of the front connection surface 571 to the center portion thereof, the front outer connection surface 5713 extends forward while forming a curved surface, so the center portion of the front inner connection surface 5711 and the front outer connection surface 5713 may be formed to protrude forward by having a gently curved surface. Accordingly, the contact surface of the bearing surface 51 with the talus element 1 described above is increased and thus stress is distributed and the movement of the ankle joint can be effectively reproduced. In the another embodiment of the present disclosure, when the front inner connection surface 5711 and the front outer connection surface 5713 extend to the center portion of the front connection surface 571 from the inner and outer sides thereof, respectively, the front inner connection surface 5711 and the front outer connection surface 5713 may be connected to each other in such a manner that the center portion of the front inner connection surface 5711 and the front outer connection surface 5713 is not formed as a curved surface but has a predetermined angle.

In addition, the center portion of the front connection surface 571 may be formed by protruding downward. The center portion of the upper surface of the talus element 1 is formed by being depressed downward, and to form the bearing surface 51 corresponding to the center portion of the upper surface of the talus element 1, the center portion of the bearing surface 51 is formed by protruding downward as described above. Accordingly, the center portion of a part of the front connection surface 571 connected to the front bearing boundary 58 to be described later is formed by protruding downward, compared to the remaining portion of the front inner connection surface 5711 and the front outer connection surface 5713. Accordingly, the lower end Q1 of the center portion of the front connection surface 571 is defined as the lowermost end of the front connection surface 571.

In front of the bearing element 5, the rear connection surface 573 is formed to connect the rear surface 553 with the bearing surface 51 and is formed to have a predetermined curvature and have a predetermined angle A2 between the rear connection surface 573 and the rear surface 553 of the peripheral surface 55. The rear connection surface 573 includes a rear inner connection surface 5731 and a rear outer connection surface 5733.

The rear inner connection surface 5731 is bordered by the inner bearing surface 511 through the rear bearing boundary 59 to be described later. The inner bearing surface 511 indicates a bearing surface formed on a portion close to the center of a human body relative to the AP line crossing the center of the bearing element 5, so the rear inner connection surface 5731 is defined as a rear connection surface formed on a portion close to the center of a human body.

The rear outer connection surface 5733 is bordered by the outer bearing surface 513 through the rear bearing boundary 59. Accordingly, the rear outer connection surface 5733 indicates a rear connection surface formed on a portion close to the outer side of a human body relative to the AP line.

The rear inner connection surface 5731 and the rear outer connection surface 5733 extend respectively from the inner and outer sides of the rear connection surface to the center portion of the bearing element 5 on which the AP line thereof is formed so as to be connected with each other in the center portion. In this case, the center portion on which the rear inner connection surface 5731 and the rear outer connection surface 5733 are connected with each other may be formed to protrude rearward. A mechanism in which the center portion of the rear connection surface 573 is formed by protruding rearward and downward is the same as a mechanism in which the center portion of the front connection surface 571 is formed by protruding forward and downward, so the description of the center portion of the rear connection surface 573 is replaced with the description of the center portion of the front connection surface 571.

Accordingly, the center portion of the rear connection surface 573 is formed by protruding rearward, so the contact area of the bearing element 5 with the talus element 1 is maximized such that stress can be effectively distributed and the break and wear of the bearing element can be prevented, and when the rear inner connection surface 5731 and the rear outer connection surface 5733 are connected with each other in the center portion thereof, the center portion may be formed to gently protrude rearward by having a curved surface. In addition, the center portion of the rear connection surface 573 is formed by protruding downward, so the lower end Q2 of the center portion is defined as the lower end of the rear connection surface 573.

Referring back to FIGS. 7 and 8, the front bearing boundary 58 is formed on the lower end of the front connection surface 571 so as to form a boundary between the bearing surface 51 and the front connection surface 571. Since the bearing surface 51 and the front connection surface 571 are formed as curved surfaces, the front bearing boundary 58 may also be preferably formed as a curved line. The front bearing boundary 58 may include a front inner bearing boundary 581 and a front outer bearing boundary 583.

The front inner bearing boundary 581 may be formed to define the front inner connection surface 5711 by forming a boundary between the front connection surface 571 and the inner bearing surface 511. Accordingly, the front inner bearing boundary 581 indicates a front bearing boundary formed on the center of a human body relative to the AP line.

The front outer bearing boundary 583 forms a border between the front connection surface 571 and the outer bearing surface 513, and preferably forms a border between the front outer connection surface 5713 and the outer bearing surface 513. Accordingly, the front outer bearing boundary 583 is defined as a front bearing boundary formed close to the outer side of a human body.

The front inner bearing boundary 581 and the front outer bearing boundary 583 extend respectively from the inner and outer sides of the front bearing boundary to the center portion of the bearing element 5 on which the AP line thereof is formed so as to be connected with each other and form a boundary between the center portion of the front connection surface 571 and the center portion of the bearing surface 51. The center portion of the front connection surface 571 is formed by protruding forward and/or downward, and the center portion of the bearing surface 51 is formed by protruding downward, and the center portion of the front bearing boundary 58 may be preferably formed in the shape of a curved line by protruding downward between the front inner bearing boundary 581 and the front outer bearing boundary 583. Accordingly, referring to FIG. 12, the lowermost end Q1 of the front connection surface 571 may be formed on the center portion of the front bearing boundary 58, and height between the tibial element contact surface 53 and the lowermost end Q1 of the front connection surface may be defined as H1.

In the exemplary embodiment of the present disclosure, the inner bearing surface 511 and the outer bearing surface 513 extend respectively from the inner and outer sides of the bearing surface 513 to the center portion of the bearing surface, and the connecting center portion of the inner bearing surface 511 and the outer bearing surface 513 protrudes downward by having a gently curved surface. Since the front bearing boundary 58 is formed as a curved line, the center portion of the front bearing boundary 58 protrudes downward by having a gently curved line. However, in the another embodiment of the present disclosure, the center portion of the bearing surface 51 may be connected by having a predetermined angle without having a curved surface, and in this case, the center portion of the front bearing boundary 58 may also be connected by having a predetermined angle without having a curved line.

The rear bearing boundary 59 is formed on the lower end of the rear connection surface 573 so as to form a border between the bearing surface 51 and the rear connection surface 573. Since the bearing surface 51 and the rear connection surface 573 are formed as curved surfaces, the rear bearing boundary 59 may be preferably formed as a curved line. The rear bearing boundary 59 may include a rear inner bearing boundary 591 and a rear outer bearing boundary 593.

The rear inner bearing boundary 591 may form a border between the rear connection surface 573 and the inner bearing surface 511 to define the rear inner connection surface 5731. Accordingly, the rear inner bearing boundary 591 indicates a rear bearing boundary formed on the central of a human body relative to the AP line.

The rear outer bearing boundary 593 forms a boundary between the rear connection surface 573 and the outer bearing surface 513, and is preferably formed to form a border between the rear outer connection surface 5733 and the outer bearing surface 513. Accordingly, the rear outer bearing boundary 593 is defined as a rear bearing boundary formed close to the outer side of a human body.

The rear inner bearing boundary 591 and the rear outer bearing boundary 593 extend respectively from the inner and outer sides of the rear bearing boundary to the center portion of the bearing element 5 on which the AP line thereof is formed so as to be connected with each other in the center portion and form a boundary between the center portion of the rear connection surface 573 and the center portion of the bearing surface 51. The center portion of the rear connection surface 573 is formed by protruding rearward and/or downward, and the center portion of the bearing surface 51 is formed by protruding downward. The center portion of the rear bearing boundary 59 may be preferably formed in the shape of a curved line by protruding downward between the rear inner bearing boundary 591 and the rear outer bearing boundary 593. Accordingly, referring to FIG. 12, the lowermost end Q2 of the rear connection surface 573 may be formed on the center portion of the rear bearing boundary 59, and height between the tibial element contact surface 53 and the lowermost end Q2 of the rear connection surface may be defined as H2.

The center portion of the rear bearing boundary 59 preferably protrudes downward by having a gently curved line as in the case of the front bearing boundary 58. However, in the another embodiment of the present disclosure, the center portion of the bearing surface 51 may be connected by having a predetermined angle without forming a curved surface, and in this case, the center portion of the rear bearing boundary 59 may also be connected by having a predetermined angle without having a curved line.

Hereinafter, with reference to FIG. 12, the comparison of the front connection surface 571 with the rear connection surface 573 will be described. According to the exemplary embodiment of the present disclosure, the front connection surface 571 and the rear connection surface 573 are formed asymmetrically. Specifically, height between the tibial element contact surface 53 and the lowermost end Q1 of the front connection surface 571 is different from height between the tibial element contact surface 53 and the lowermost end Q2 of the rear connection surface 573. It is preferable that height between the tibial element contact surface 53 and the lowermost end Q2 of the rear connection surface 573 is smaller than height between the tibial element contact surface 53 and the lowermost end Q1 of the front connection surface 571 (H1>H2). It is more preferable that the heights of the front surface 551 and the rear surface 553 extending from the tibial element contact surface 53 are the same, but height between a boundary between the rear surface 553 and the rear connection surface 573 and the center portion of the rear bearing boundary 59 to be described later is smaller than height between a boundary between the front surface 551 and the front connection surface 571 and the center portion of the front bearing boundary 58 to be described later. In addition, an angle formed between the rear connection surface 573 and the rear surface 553 may be smaller than an angle formed between the front connection surface 571 and the front surface 551 (A1>A2).

As described above, in artificial ankle joint replacement, each element of the artificial ankle joint is inserted and mounted from the anterior side of an ankle. After inserting the talus element 1 and the tibial element 3, the bearing element 5 is inserted into space therebetween, and when the front and rear of the bearing element 5 are symmetrical to each other, the stable insertion and fixing of the bearing element 5 is difficult. Accordingly, in the exemplary embodiment of the present disclosure, height between the tibial element contact surface 53 and the lowermost end Q2 of the rear connection surface 573 is smaller than height between the tibial element contact surface 53 and the lowermost end Q1 of the front connection surface 571 (H1>H2) such that the insertion of the bearing element 5 is easy. In addition, in the exemplary embodiment of the present disclosure, to minimize interference with the protruding portion of the rear during the insertion of the bearing element 5, an angle formed between the rear connection surface 573 and the rear surface 553 may be smaller than an angle formed between the front connection surface 571 and the front surface 551 (A1>A2).

The above description has been limited to the bearing element used in the artificial ankle joint, but is applicable even to an implant used in an artificial knee joint, an artificial hip joint, and an artificial shoulder joint.

The above detailed description is intended to illustrate the present disclosure. In addition, the foregoing is intended to represent and describe the exemplary embodiments of the present disclosure, and the present disclosure may be used in various other combinations, variations, and environments. That is, changes or modifications are possible within the scope of the concept of the invention disclosed herein, the scope equivalent to the written disclosure, and/or within the scope of skill or knowledge in the art. The written embodiments are intended to describe the best state for embodying the technical spirit of the present disclosure, and various changes required in specific application field and use of the present disclosure are also possible. Accordingly, the above detailed description is not intended to limit the present disclosure to the disclosed embodiments. In addition, the appended claims should be construed to include other embodiments as well.

1. An implant implanted in a body through artificial ankle joint replacement, the implant being a bearing element which enables a movement of an ankle between a talus element and a tibial element, the bearing element comprising:
a bearing surface which enables a movement of the bearing element on an articular surface of the talus element while the bearing element is in contact with the talus element;
a peripheral surface formed to surround a side surface of the bearing element; and
a connection surface which connects the peripheral surface with the bearing surface.

2. The implant of claim 1, wherein the connection surface comprises a front connection surface formed on a front of the bearing element, wherein the front connection surface is formed by having a predetermined angle from the peripheral surface.

3. The implant of claim 2, wherein the front connection surface comprises: a front inner connection surface bordered by an inner bearing surface and a front outer connection surface bordered by an outer bearing surface,
wherein the front inner connection surface and the front outer connection surface extend respectively from inner and outer sides of the front connection surface such that a center of the front inner connection surface and the front outer connection surface protrudes forward, so that a contact area of the bearing surface with the talus element is increased.

4. The implant of claim 3, wherein the front inner connection surface and the front outer connection surface are formed by having curvatures and extend respectively from the inner and outer sides of the front connection surface such that the center of the front inner connection surface and the front outer connection surface protrudes by having a curved surface.

5. The implant of claim 2, wherein the connection surface further comprises a rear connection surface formed on a rear of the bearing element,
wherein the rear connection surface comprises: a rear inner connection surface bordered by an inner bearing surface and a rear outer connection surface bordered by an outer bearing surface, wherein the rear inner connection surface and the rear outer connection surface extend respectively from inner and outer sides of the rear connection surface such that a center of the rear inner connection surface and the rear outer connection surface protrudes rearward, so that a contact area of the bearing surface with the talus element is increased.

6. The implant of claim 5, wherein the rear inner connection surface and the rear outer connection surface are formed by having curvatures and extend respectively from the inner and outer sides of the rear connection surface such that the center of the rear inner connection surface and the rear outer connection surface protrudes by having a curved surface.

7. The implant of claim 2, wherein the front connection surface comprises: a front inner connection surface bordered by an inner bearing surface; and a front outer connection surface bordered by an outer bearing surface, wherein the front inner connection surface and the front outer connection surface extend respectively from inner and outer sides of the front connection surface such that a center of the front inner connection surface and the front outer connection surface protrudes forward, and
a rear connection surface comprises: a rear inner connection surface bordered by the inner bearing surface, and a rear outer connection surface bordered by the outer bearing surface, wherein the rear inner connection surface and the rear outer connection surface extend respectively from inner and outer sides of the rear connection surface such that a center of the rear inner connection surface and the rear outer connection surface protrudes rearward, so that a contact area of the bearing surface with the talus element is increased.

8. The implant of claim 7, wherein the front inner connection surface and the front outer connection surface are formed by having curvatures and extend respectively from the inner and outer sides of the front connection surface such that the center of the front inner connection surface and the front outer connection surface protrudes by having a curved surface, and the rear inner connection surface and the rear outer connection surface extend respectively from the inner and outer sides of the rear connection surface such that the center of the rear inner connection surface and the rear outer connection surface protrudes by having a curved surface.

9. The implant of claim 1, wherein the bearing element further comprises a tibial element contact surface formed to be in contact with the tibial element, and the peripheral surface comprises a front surface formed on a front of the bearing element and a rear surface formed on a rear of the bearing element.

10. The implant of claim 9, wherein a front boundary which forms a boundary between the front surface and the tibial element contact surface is formed in a form of an arc by being convex forward, and the tibial element contact surface is formed by extending forward up to the front boundary.

11. The implant of claim 9, wherein a rear boundary which forms a boundary between the rear surface and the tibial element contact surface is formed in a form of an arc by being convex rearward, and the tibial element contact surface is formed by extending rearward up to the rear boundary.

12. The implant of claim 9, wherein a front boundary which forms a boundary between the front surface and the tibial element contact surface is formed in a form of an arc by being convex forward, and the tibial element contact surface is formed by extending forward up to the front boundary, and
a rear boundary which forms a boundary between the rear surface and the tibial element contact surface is formed in a form of an arc by being convex rearward, and the tibial element contact surface is formed by extending rearward up to the rear boundary.

13. An implant implanted in a body through artificial ankle joint replacement, the implant being a bearing element which enables a movement of an ankle between a talus element and a tibial element, the bearing element comprising:
a front and a rear which are formed asymmetrically.

14. The implant of claim 13, wherein the bearing element comprises: a bearing surface which enables a movement of the bearing element on an articular surface of the talus element while the bearing element is in contact with the talus element; and a tibial element contact surface formed to be in contact with the tibial element,
wherein a height of the rear between the tibial element contact surface and the bearing surface is smaller than a height of the front between the tibial element contact surface and the bearing surface.

15. The implant of claim 14, wherein the bearing element further comprises: a peripheral surface formed to surround a lateral surface of the bearing element; and a connection surface which connects the peripheral surface with the bearing surface,
wherein the connection surface comprises a front connection surface formed on the front of the bearing element and a rear connection surface formed on the rear of the bearing element, each of the front connection surface and the rear connection surface being formed by having a predetermined angle from the peripheral surface.

16. The implant of claim 15, wherein a height between the tibial element contact surface and a lowermost end of the rear connection surface is smaller than a height between the tibial element contact surface and a lowermost end of the front connection surface.

17. The implant of claim 16, further comprising:
a front bearing boundary which is formed on a lower end of the front connection surface and forms a boundary between the bearing surface and the front connection surface; and
a rear bearing boundary which is formed on a lower end of the rear connection surface and forms a boundary between the bearing surface and the rear connection surface.

18. The implant of claim 17, wherein the front bearing boundary comprises: a front inner bearing boundary which forms a boundary between the front connection surface and an inner bearing surface; and a front outer bearing boundary which forms a boundary between the front connection surface and an outer bearing surface, wherein the front inner bearing boundary and the front outer bearing boundary extend respectively from inner and outer sides of the front bearing boundary such that a center of the front inner bearing boundary and the front outer bearing boundary protrudes downward to form the lowermost end of the front connection surface, and
the rear bearing boundary comprises: a rear inner bearing boundary which forms a boundary between the rear connection surface and the inner bearing surface; and a rear outer bearing boundary which forms a boundary between the rear connection surface and the outer bearing surface, wherein the rear inner bearing boundary and the rear outer bearing boundary extend respectively from inner and outer sides of the rear bearing boundary such that a center of the rear inner bearing boundary and the rear outer bearing boundary protrudes downward to form the lowermost end of the rear connection surface.

19. The implant of claim 18, wherein the peripheral surface comprises: a front surface formed on the front of the bearing element; and a rear surface formed on the rear of the bearing element,
wherein a height of the front surface extending vertically from the tibial element contact surface is the same as a height of the rear surface extending vertically from the tibial element contact surface, and
a height between a boundary between the rear surface and the rear connection surface and the lowermost end of the rear connection surface is smaller than a height between a boundary between the front surface and the front connection surface and the lowermost end of the front connection surface.

20. The implant of claim 18, wherein the front connection surface comprises: a front inner connection surface bordered by the inner bearing surface; and a front outer connection surface bordered by the outer bearing surface, wherein the front inner connection surface and the front outer connection surface extend respectively from inner and outer sides of the front connection surface such that a center of the front inner connection surface and the front outer connection surface protrudes forward, and
the rear connection surface comprises: a rear inner connection surface bordered by the inner bearing surface, and a rear outer connection surface bordered by the outer bearing surface, wherein the rear inner connection surface and the rear outer connection surface extend respectively from inner and outer sides of the rear connection surface such that a center of the rear inner connection surface and the rear outer connection surface protrudes rearward, so that a contact area of the bearing surface with the talus element is increased.
